(19) United States
(12) Patent Application Publication
(10) Pub. No.: US 2014/0147523 A1
(43) Pub. Date: May 29, 2014

(54) EXTRACTS OF SOLANUM ESURIALE AND SOLANUM GLAUCOPHYLLUM AND METHODS OF TREATING SKIN

(71) Applicants: Khalid Mahmood, South Hadley, MA (US); Kurt Reynertson, Hopewell, NJ (US); Meghan Russell, Lawrenceville, NJ (US); Michael Southall, Pennington, NJ (US)

(72) Inventors: Khalid Mahmood, South Hadley, MA (US); Kurt Reynertson, Hopewell, NJ (US); Meghan Russell, Lawrenceville, NJ (US); Michael Southall, Pennington, NJ (US)

(73) Assignee: Johnson & Johnson Consumer Companies, Inc., Skillman, NJ (US)

(21) Appl. No.: 14/090,061

(22) Filed: Nov. 26, 2013

Related U.S. Application Data
(60) Provisional application No. 61/730,557, filed on Nov. 28, 2012.

(51) Int. Cl. A61K 8/97; A61Q 17/00; A61Q 19/08; A61Q 19/00

(52) U.S. Cl. CPC A61K 8/97; A61Q 19/007; A61Q 17/00; A61Q 19/08
USPC 424/725

(57) ABSTRACT

The present invention relates to compositions comprising extracts of Solanum esuriale and/or Solanum glaucophyllum and methods of treating skin, e.g, dry skin, xerotic skin, skin having a compromised barrier, or skin in need of anti-aging treatment using said compositions.

### FIELD OF THE INVENTION

The present invention relates to compositions comprising, as well as methods of using compositions comprising, extracts of Solanum esuriale, Solanum glaucophyllum, or both. The compositions are useful for example for treating skin in need of treatment for dryness, e.g., dry skin, skin having a compromised barrier, or skin in need of anti-aging treatment.

### SUMMARY OF THE INVENTION

The present invention relates to a topical composition comprising a cosmetically-acceptable topical carrier, wherein the carrier comprises an ingredient selected from water, a humectant, an emollient, and combinations thereof; and an extract of Solanum esuriale.

The present invention also relates to a topical composition comprising a cosmetically-acceptable topical carrier, wherein the carrier comprises an ingredient selected from water, a humectant, an emollient, and combinations thereof; and an extract of Solanum glaucophyllum.

The invention also relates to a method treating skin, e.g., dry skin or skin having a compromised barrier. The method includes topically applying to said skin a composition comprising an extract of one or both of Solanum esuriale and Solanum glaucophyllum. According to certain embodiments, a method of treating skin includes applying to skin in need of treatment for dryness a composition comprising an extract of Solanum esuriale.

Other features and advantages of the present invention will be apparent from the detailed description of the invention and from the claims.

### DETAILED DESCRIPTION OF PREFERRED EMBODIMENTS

Unless defined otherwise, all technical and scientific terms used herein have the same meaning as commonly understood by one of ordinary skill in the art to which the invention belongs. Also, all publications, patent applications, patents, and other references mentioned herein are incorporated by reference. Unless otherwise indicated, a percentage refers to a percentage by weight (i.e., % (W/W). Unless stated otherwise, all ranges are inclusive of the endpoints, e.g., “from 4 to 9” includes the endpoints 4 and 9.

As used herein, “treating” refers to mitigating, reducing, preventing, improving, or eliminating the presence or appearance of a condition or disease.

As used herein, “cosmetic” refers to a beautifying substance or preparation which preserves, restores, bestows, simulates, or enhances the appearance of bodily beauty or appears to enhance the beauty or youthfulness, specifically as it relates to the appearance of tissue or skin.

As used herein, “cosmetically acceptable” means suitable for use in contact with (human) tissues (e.g., the skin) without undue toxicity, incompatibility, instability, irritation, allergic response, and the like, commensurate with a reasonable benefit/risk ratio.

It is believed that one skilled in the art can, based upon the description herein, utilize the present invention to its fullest extent. The following specific embodiments are to be construed as merely illustrative, and not limitative of the remainder of the disclosure in any way whatsoever.

Solanum glaucophyllum (S. malacoxylon, waxyleaf nightshade) is a perennial shrub/rhizomatous plant with a simple stem and shortly branched. Typically leaves are simple, ovate, lanceolate, greenish-gray, 1-2 cm long, and it may have bluish purple flowers. The fruit is typically a globose berry 1-2 cm in diameter, blue-black, and features several seeds inside.

Solanum esuriale (potato bush, wild tomato, Quena, potato weed) is clonal perennial herb that may grow up to 30 cm, typically has a grey-green or pale green color, densely pubescent with stellate hairs. Prickles are usually absent, but sometimes present towards base of plant. Berries are globular in shape, 10-15 mm diam., pale yellow or light yellow-brown. Seeds are 2-3 mm long, pale yellow-brown. Solanum esuriale is native to New South Wales, Australia. It occurs in many habitats in clay, clay-loam, and loam, often along seasonal pools, creek-lines or levee banks of inland streams) in near-coastal sandy soils.

As used herein the term “extracts of Solanum esuriale and/or Solanum glaucophyllum” refers to compositions in which the either or both of Solanum esuriale and/or Solanum glaucophyllum has been harvested and or one or more parts thereof (e.g., flower, seed, root, rhizome, stem, fruit and/or leaf of the plant) have been optionally isolated. The plant may be further prepared by finely dividing the harvested plant or parts thereof, such as by crushing, grinding or milling to a finely divided solid or powder. The plant or portions thereof are preferably contacted with a solvent, e.g., steam, water that may or not be heated; or other solvents to extract portions thereof. A suitable extraction process may comprise contacting the one or more parts of the plant with a solvent system (e.g., water, glycol, and or alcohol).

### Topical Compositions

The extracts of Solanum esuriale and/or Solanum glaucophyllum may be combined with one or more cosmetically acceptable carriers to form a composition (e.g., formulation) suitable for use on visibly dry skin.

Suitable carriers of this invention include, but are not limited to, water, as well as humectants such as diols or other polyols including 1,2-propanediol, glycerin, propylene glycol, butylene glycol, hexylene glycol, polyethylene glycol (PEG), and combinations thereof. While in certain embodiments lower alcohols such as ethanol and isopropanol may be included, in other embodiments, in order to reduce likelihood of sky drying, these alcohols are excluded from the composition (e.g., present in less than 0.5%)

In order to facilitate treatment of dry skin, in certain embodiments of the invention the cosmetically acceptable carrier includes both a humectant as well as an emollient. By emollient it is meant cosmetic ingredients that are generally insoluble in water and suitable for “leave on” applications for the skin. Suitable emollients include hydrophobic compounds useful to prevent transepidermal loss of water from the skin. Examples of emollients include hydrophobic compounds such as vegetable oils, mineral oils (e.g., petrolatum), fatty esters (e.g., esters such as isopropyl palmitate, isopropyl myristate, isononyl isonanoate) including those fatty esters of glycerol, silicone oils (e.g., dimethicone), waxes (various waxy hydrocarbons—straight or branched chain alkanes or alkenes, ketone, diketone, primary or secondary alcohols, aldehydes, sterol esters, alkanoic acids, turpenes, monoesters, such as those having a carbon chain length ranging from C₁₂-C₃₈), and the like.

In order to facilitate the formulation of a suitable cosmetically-acceptable carrier, one may include any of various functional ingredients in the composition. For example, one may include any of a number of sequesterants, emulsifiers, thickeners, polymers, preservatives, colorants, fragrances, antioxidants, and other ingredients commonly used in personal care and cosmetic products. Suitable emulsifiers include, for example, non-ionic emulsifiers such as fatty alcohols including polyethoxylated fatty alcohols, esters of glycerol; anionic emulsifiers such as alkyl phosphates; cationic emulsifiers such as alkyl quaternary ammonium compounds. Suitable thickeners include hydrophobically modified acrylic polymers, cellulose polymers and clays.

While in certain embodiments (in which the product is in the form of a cleanser) cleansing surfactants such as betaines, sulfates, sulfonates, polyglycosides, among other wetting agents typically utilized for wetting and foam generation may be included, in other embodiments, in order to reduce likelihood of sky drying and to make the product suitable for a “leave-on” application, cleansing surfactants are excluded from the composition.

The cosmetically acceptable carrier in the topical composition may constitute from about 40% to about 99.99%, by weight, of the composition, more preferably from about 80% to about 95%, by weight, of the composition.

In one embodiment, the compositions according to this invention may further contain one or more additional cosmetically active agent(s) as well as the above-mentioned components. What is meant by a “cosmetically active agent” is a compound, which may be a synthetic compound or a compound extracted, isolated, purified or concentrated from a natural source, or a natural extract containing a mixture of compounds, that has a cosmetic or therapeutic effect on the tissue, including, but not limited to: anti-microbial agents such as anti-yeast, anti-fungal, and anti-bacterial agents, anti-inflammatory agents, anti-aging agents, depigmentaion agents, anti-parasite agents, antioxidants, ant-acne agents, keratolytic agents, nutrients, vitamins, minerals, sunscreens, sebum modulators, anti-cellulite agents and the like.

The extracts of Solanum esuriale and/or Solanum glaucophyllum and cosmetically acceptable topical carrier and optional additional cosmetically active agents may be combined in any proportion to form a composition suitable for topical use. In one embodiment of the invention, the topical composition includes at least about 0.1% of the extracts of Solanum esuriale and/or Solanum glaucophyllum. In certain embodiments the composition includes at least about 0.5%, and in certain other embodiments from about 0.5% to about 5%, such as from about 1% to about 4% of the extracts of Solanum esuriale and/or Solanum glaucophyllum.

The cosmetically-acceptable carrier and the extracts of Solanum esuriale and/or Solanum glaucophyllum may be formulated into any of various product types including, but not limited to solutions, suspensions, emulsions such as microemulsions and nanoemulsions, gels, solids and liposomes. The compositions may be made into a wide variety of cosmetic articles that include but are not limited to lotions, creams, gels, sticks, sprays, ointments, cleansing liquid washes and solid bars, pastes, foams, powders, mousses, wipes, strips, patches, wound dressing and adhesive bandages, hydrogels, film-forming products, as well as facial and skin masks. Other forms can be formulated by those of ordinary skill in the art.

Compositions comprising extract may be topically applied to mammalian skin that is in need of treatment for dryness, i.e., dry, xerotic (dry or dehydrated skin that shows visible signs of flaking), skin having compromised bather properties and/or skin in need of anti-aging treatment, in order to treat the face or body, including the hands, feet, scalp, elbows, knees, ankles, nape of the neck, among other areas portions of the skin. In certain other embodiments, topical compositions comprising extracts of Solanum esuriale and/or Solanum glaucophyllum are topically applied to dry skin or skin having compromised barrier properties, or skin characterized by certain disease states such as eczematic skin, psoriatic skin, or skin characterized by atopic dermatitis. In certain other embodiments, topical compositions comprising extracts of Solanum esuriale and/or Solanum glaucophyllum are topically applied to skin in need of anti-aging treatment including skin exhibiting signs of aging, such as lines, wrinkles, loss of elasticity, sagging, skin dryness and unevenness, blotches, and age spots. The compositions may be applied to the skin in need of such treatment according to a suitable treatment regimen, e.g., every month, every week, every other day, every day, twice a day, or the like.

It is believed that one skilled in the art can, based upon the description herein, utilize the present invention to its fullest extent.

What is claimed is:

1. A topical composition, comprising:
a cosmetically-acceptable topical carrier, wherein the carrier comprises an ingredient selected from water, a humectant, an emollient, and combinations thereof; and
an extract of Solanum esuriale.

2. The topical composition of claim 1, wherein the carrier comprises both a humectant and an emollient.

3. The topical composition of claim 1, wherein the humectant is a polyol.

4. The topical composition of claim 1, wherein the emollient is selected from vegetable oils, mineral oils, fatty esters, silicone oils, waxes, and combinations thereof

5. A method of treating skin, comprising topically applying to skin in need of treatment for dryness the composition of claim 1.

6. The method of claim 1, wherein the skin is dry skin, xerotic skin, skin having a compromised barrier, or skin in need of anti-aging treatment.
